(19) United States
(12) Patent Application Publication
(10) Pub. No.: US 2023/0038398 A1
(43) Pub. Date: Feb. 9, 2023

(54) SYSTEM AND METHOD FOR USING A DIGITAL VIRTUAL SPONSOR FOR BEHAVIORAL HEALTH AND WELLNESS OF A USER

(71) Applicant: Addiction Resource Systems, Inc., Aventura, FL (US)

(72) Inventors: Hayes THOMAS, Aventura, FL (US); Matthew Charles REEDY, San Antonio, TX (US)

(73) Assignee: Addiction Resource Systems, Inc., Aventura, FL (US)

(21) Appl. No.: 17/970,269

(22) Filed: Oct. 20, 2022

Related U.S. Application Data
(63) Continuation-in-part of application No. 16/441,886, filed on Jun. 14, 2019.
(60) Provisional application No. 62/685,047, filed on Jun. 14, 2018.

(51) Int. Cl. G16H 20/70

(52) U.S. Cl. CPC G16H 20/70

(57) ABSTRACT

A method and system pertaining to a digital virtual sponsor to facilitate behavioral health and wellness of a user is provided. The method includes receiving an input from the user requiring assistance for a behavioral health issue, analyzing the input from the user by using an emotion recognition process to generate data associated with the user, storing the data associated with the user for access at a later time, providing the user with an instruction to complete a task as part of a program to facilitate the behavioral health and wellness of the user on a basis of the data associated with the user, and receiving an indication that the user completed the task to indicate an adherence to the program by the user.

### CROSS-REFERENCE TO RELATED APPLICATIONS

This application is a Continuation-in-Part of copending application Ser. No. 16/441,886, filed on Jun. 14, 2019, which claims priority under 35 U.S.C. § 119(e) to U.S. Provisional Application No. 62/685,047, filed on Jun. 14, 2018, all of which are hereby expressly incorporated by reference into the present application.

### BACKGROUND OF THE INVENTION

This specification relates to using natural language processing and machine learning to help facilitate behavioral health and wellness. The term “behavioral health” in this context means the promotion of mental health, resilience and well-being; the treatment of mental and substance use disorders; and the support of those individuals who experience and/or are in recovery from these conditions, along with their families and communities.

Behavioral health and wellness programs are designed to help individuals recover from mental health conditions such as, but not limited to, addiction (substance use disorder), depression, anxiety and post-traumatic stress disorder (PTSD). These programs provide individuals with counseling, advice, accountability and incentives for taking necessary steps and actions to address their behavioral health issues. For example, a twelve-step program includes a set of guiding principles outlining steps for addiction recovery including: admitting that a person cannot control addiction; recognizing a higher power that can give strength; examining past errors with the help of a sponsor; making amends for these errors; learning to live a new life with a new behavior code; and helping others who suffer from the same addictions.

Staying on a behavioral health and wellness program can be extremely difficult for a person struggling with a mental health condition. In order to adhere to a wellness program, a person needs to stay motivated. Often, individuals with these conditions rely on others to help them with this motivation, meeting regularly with a sponsor or group of people and working through the recovery steps together.

Sometimes individuals struggling with behavioral health issues cannot feasibly meet in person with a sponsor or attend group meetings, or need more support than is provided in groups or with sponsors. These individuals need daily accountability and incentives to help them stay motivated in the recovery process.

### SUMMARY OF THE INVENTION

This specification describes technologies for using natural language processing and machine learning to help facilitate recovery from a variety of behavioral health issues. These technologies generally involve a digital virtual sponsor system and method for creating a digital virtual sponsor to hold a person struggling with a mental health disorder accountable for his or her actions and to provide incentives to encourage the person to make good choices on the steps to recovery without requiring the person to attend group meetings or meet with a sponsor in person.

In one embodiment, a digital virtual sponsor system includes a digital assistant platform that interacts with an individual using natural language processing to create a dialogue with the individual regarding positive choices and recovery behavior, a knowledge base that contains information and resources about behavioral health, and blockchain processes that use machine learning models and the knowledge base to provide the individual with personalized answers, resources, sobriety challenges, and other necessary information to facilitate recovery.

In general, one innovative aspect of the subject matter described in this specification can be embodied in methods that include the actions of creating a digital virtual sponsor for people struggling with behavioral health issues.

Other embodiments of this aspect include corresponding computer systems, apparatus, and computer programs recorded on one or more computer storage devices, each configured to perform the actions of the methods. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. In particular, one embodiment includes all the following features in combination.

The subject matter described in this specification can be implemented in particular embodiments so as to realize one or more of the following advantages.

A digital virtual sponsor system provides interaction, accountability and incentives for a person struggling with behavioral health issues without the person needing to be physically present in group meetings or with a sponsor. The system uses natural language and machine learning to provide sobriety challenges, resources, and accountability to the recovering person. The system can also provide incentives for reaching certain milestones, getting through a day without relapsing, following recovery steps, and encouragement to stay sober. The system can further reach out to the struggling person if the system determines that the person is not following his or her recovery program or at risk of relapse. The system can suggest recovery activities, provide valuable information and connect with the person's contacts in order to ensure the person's safety and recovery.

In addition to incentives, the system enables friends of the recovering person to pose recovery challenges to the recovering person. A leaderboard can also indicate the progress the recovering person is making as compared to his or her friends.

The system can also provide information to third parties, e.g., accountability courts or sponsors regarding the recovering person's successes and failures. Additionally, the system may collect high quality data for research and analytics to find root causes for relapse. The system can further provide a judgment-free resource so that the recovering person can feel comfortable reaching out to the system for information and help in the recovery process.

In one embodiment, a method and system pertaining to a digital virtual sponsor to facilitate behavioral health and wellness of a user is provided. The method comprises receiving an input from the user requiring assistance for a behavioral health issue, analyzing the input from the user by using an emotion recognition process to generate data associated with the user, storing the data associated with the user for access at a later time, providing the user with an instruction to complete a task as part of a program to facilitate the behavioral health and wellness of the user on a basis of the data associated with the user, and receiving an indication that the user completed the task to indicate an adherence to the program by the user.

In this embodiment, the system continuously collects usage data that describes the current activity and progress of the recovering person over time. The generated data associated with the user can include usage data that includes, but is not limited to, information related to sentiment and emotion, location tracking, completion of activities that were prescribed by an accountability person or organization, assessment surveys, sobriety challenges, and measures of overall improvement. This data is available for an accountability person or organization to see and review through the system, as well as through a portal on a web site that provides analytical tools for improving the recovery process.

In some circumstances, recovering persons may be required to prove their adherence to a prescribed recovery/wellness program. When the recovering person completes one or more prescribed activities or tasks using the system, a non-fungible token (NFT) is created on the blockchain ecosystem and is provided to him or her as a permanent record of completion.

If the prescribed recovery/wellness program includes required attendance at 12-step meetings, workshops, seminars, group therapy sessions or training classes, trusted officials at these events may initiate the award of an NFT to the recovering person through the blockchain ecosystem using a Proof of Attendance Protocol (POAP). Based on the immutable nature of blockchains, these tokens are proof of completion and attendance at the requisite meeting, workshop, seminar session and/or class. These issued NFTs may be shown to accountability persons or organizations to irrefutably demonstrate the person's adherence to prescribed recovery and/or wellness programs.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

The accompanying drawings, which are included to provide a further understanding of the invention and are incorporated in and constitute a part of this application, illustrate embodiments of the invention and together with the description serve to explain the principles of the invention.

FIG. 1 illustrates a digital virtual sponsor system that uses natural language processing and machine learning to facilitate virtual sponsorship with incentives and accountability.

FIG. 2 illustrates an example dialogue between a person struggling with behavioral health issues and a communication device of the digital virtual sponsor system.

FIG. 3 illustrates examples of two dialogues between a person and the system that have been tailored to the person based on user profile information and previous interactions.

FIG. 4 is a flowchart of an example process for providing a digital virtual sponsor to facilitate behavioral health and wellness.

FIG. 5 is a flowchart of an example process for determining sobriety of a person struggling with addiction.

FIG. 6 is a flowchart of an example process of using a digital virtual sponsor to facilitate behavioral health and wellness of a person.

FIG. 7 is a schematic view that illustrates natural language processing in an emotion recognition process used to facilitate behavioral health and wellness of the person.

FIG. 8 is a schematic view that illustrates the emotion recognition process used to facilitate behavioral health and wellness of the person.

Like reference numbers and designations in the various drawings indicate like elements.

### DETAILED DESCRIPTION OF CERTAIN EMBODIMENTS

The specification generally describes a digital virtual sponsor system that provides dynamic, interactive dialogue, sobriety challenges, and incentives to a person struggling with behavioral health issues.

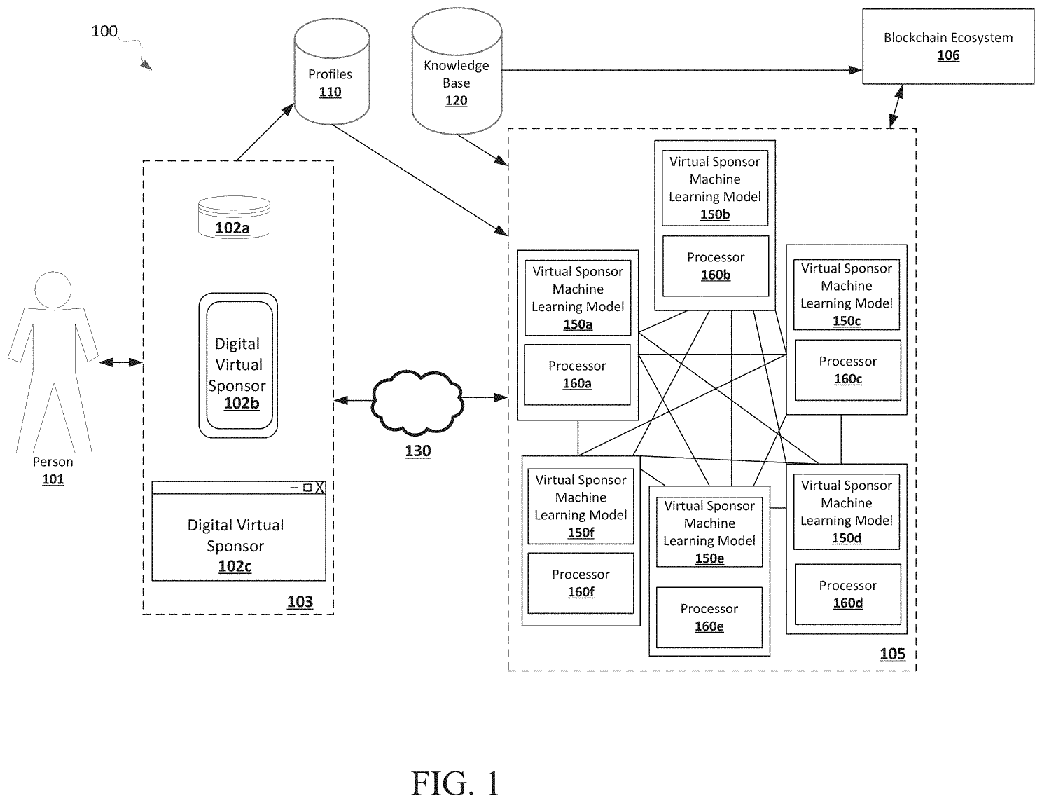

FIG. 1 illustrates an example digital virtual sponsor system 100 that engages in dynamic, interactive dialogue with a person struggling with addiction to support the person's recovery, provide accountability, and incentivize the person to make good choices. The digital virtual sponsor system 100 is an example of a system implemented as computer programs on one or more computers in one or more locations, in which the systems, components, and techniques described below can be implemented.

In some implementations, a person struggling with addiction 101 registers with the digital virtual sponsor system 100, e.g., using a web browser navigated to a web page of the digital virtual sponsor system 100 or communicates in a batch form or iteratively over time with a digital assistant to complete a profile. The person 101 can access the web page using a computing device 102c. During registration, the person 101 provides basic information to create a user profile within the system 100. The information provided by the person 101 may include: name, age, family conditions, address, contact information, emergency contact information, faith affiliation, addiction struggle, and sobriety time. Other information can be added to the user's profile, either at the time of initial creation or as the user interacts with the system 100. This information includes: information pertaining to addiction that is categorized by a recovery program and information provided by the user's victims, family, friends, and community.

Once the person 101 is registered with the system 100, the person 101 can use one or more connected devices including Internet of Everything (IoE) devices using a communication platform 103 to access the system 100 in order to obtain support for his or her recovery process. In some implementations, the person 101 accesses the digital virtual sponsor system 100 using e.g., a smartphone 102b, a computing device 102c, or a smart home device, e.g., a digital assistant, 102a. Although FIG. 1 illustrates a number of devices, other devices such as tablets, smart devices, a desktop or laptop computer, a mobile device, a wearable device, e.g., a virtual reality headset, home and building automation devices, a gas station pump equipped with digital assistant technology support, or other networked devices can facilitate interaction between the person 101 and the digital virtual sponsor system 100.

A person 101 can interact with the digital virtual sponsor system 100 by, e.g., typing, texting, using a touch screen, or conversing with the system 100 using natural language.

In one implementation, the person 101 can query the system 100 using natural, conversational language. The communication platform 103 uses a digital assistant, e.g., Amazon Alexa, Siri® from Apple, Google Home®, Microsoft Cortana®, or any one of various others, to process the natural language and understand the person's inquiry. In some instances, the query can be in the form of a question, e.g., “Alexa, what time is the recovery meeting tonight?” In other instances, the query can be a statement, e.g., “Hey Google, I think I need a drink.”

Regardless of the form of the inquiry, the system 100 receives the query from the person 101 using the communication platform 103 and processes the query using a machine learning subsystem 105 that includes virtual machine learning response models 150a-150f and processors 160a-160f for determining appropriate responses. The machine learning models are trained on example conversations with users to determine the intent of the users and the proper category for response. For example, the question “Alexa, what time is the recovery meeting tonight?” may be categorized as an information query whereas the statement “Hey Google, I think I need a drink” may be categorized as a craving or warning signs inquiry, depending on follow-on conversation with the user.

The models then predict the proper response to user queries given a predefined set of steps for different response categories. Each response category can have an initial defined template that includes the response steps. These steps can evolve and adapt to each person individually, so that the system 100 gains more information regarding people over time. For example, one recovering person may respond better to mediations while another recovering person may respond better to breathing exercises. The system learns behavior of each user and tailors its responses to each user's individual preferences and responses.

In some implementations, the machine learning subsystem 105 includes different types of machine learning models, e.g., one for natural language processing, a second type for determining an appropriate response to received natural language, and/or one for predicting whether a user will relapse. For example, the machine learning subsystem 105 may be WingMan as described in U.S. Patent Application No. 62/827,615, for DIGITAL VIRTUAL SPONSOR, which was filed on Apr. 1, 2019, and which is incorporated here by reference.

In some implementations, the machine learning models 150a-150f are neural networks. Neural networks are machine learning models that employ one or more layers of neurons to generate an output, e.g., one or more classifications, for a received input. Neural networks may include one or more hidden layers in addition to an output layer. The output of each hidden layer can be used as input to the next layer of the network, i.e., the next hidden layer or the output layer, and connections can also bypass layers, or return within the same layer such as in the case of a recurrent network. Each layer of the neural network generates an output from its inputs in accordance with the network architecture and a respective set of parameters for the layer.

In addition to typical weights and biases, networks may include gates to hold memory as well as gates to remove data from memory such as in a Long Short-Term Memory (LSTM) network. A stateful network such as the LSTM aids in sequence classification and allows the network to understand the context of current data based on prior events.

The machine learning subsystem 105 may use machine learning libraries to develop and train the machine learning models 150a-150f. For example, the system 100 may incorporate Rasa NLU and tensorflow to determine user intents and how user messages or queries should be categorized for natural language processing machine learning models and to develop and train response and predictive models.

The machine learning subsystem 105 uses information from a knowledge base 120 and user profiles 110 to train response and relapse-predictive models to provide responsive dialogue for user questions and issues related to overcoming addiction issues. The knowledge base 120 is created through (1) public information on addiction and resources, e.g., currently available information from books, websites, recovery program material, etc.; (2) private information on addiction and resources, e.g., information from physicians, academic researchers, sponsors, group members, family members, and other addicts; and (3) machine-generated information from computer applications. Information includes advice, recommendations, guidance, and interactions that have been proven helpful previously to recovering persons and that may be valuable in a current context, e.g., a conversation with a person struggling with addiction.

The system 100 additionally includes a blockchain ecosystem 106 that collects and tracks the progresses of users and information in the recovery process. The blockchain ecosystem 106 supports non-fungible tokens (NFTs) to represent an accomplishment of a behavioral health and wellness program task by person 101. NFTs indicate attendance by person 101 at a specific event, meeting, conference, therapy session, etc., using the Proof of Attendance Protocol (POAP). In addition, NFTs indicate proof of completion of behavioral health and wellness program tasks by person 101. In these embodiments, the NFTs are digital assets that are held in a digital wallet owned and controlled by person 101. In one embodiment, NFTs are a record of a person's life experience and can be used as a memento. Also, when requested by an accountability court, a supervising organization (such as one that is providing a behavioral health and wellness program) or other responsible party, person 101 may display the NFT record of completion or attendance of a task, meeting, program or the like, using communication device 102a, smartphone 102b and/or computing device 102c. Since the blockchain ecosystem 106 is immutable, any NFT record stored therein is irrefutable evidence of attendance or completion of a task, meeting, program or the like. In some implementations, the information stored on the blockchain ecosystem 106 can be used to train the machine learning models 150a-150f in the machine learning subsystem 105.

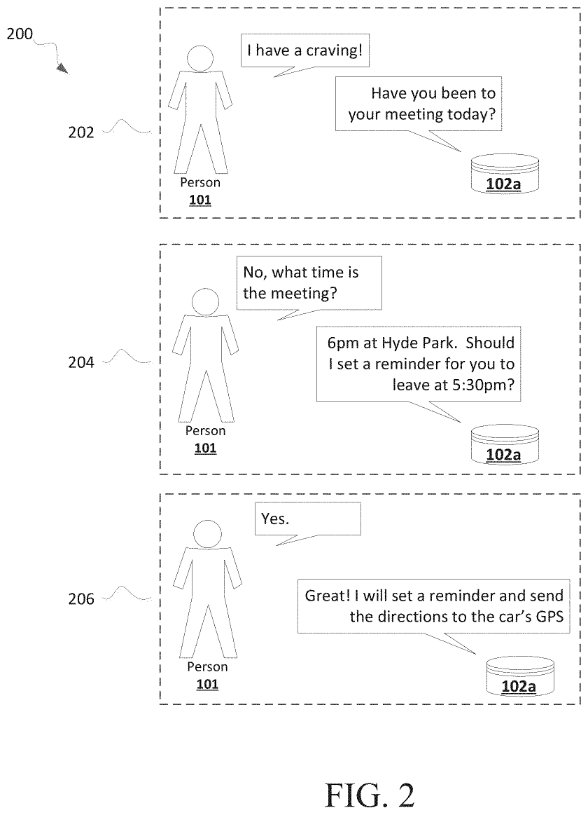

FIG. 2 illustrates an example dialogue between a person struggling with addiction and a communication device 102a of the digital virtual sponsor system 100. The dialogue 200 is broken into three interactions 202-206 of back and forth dialogue between the person 101 and the communication device 102a. In a first interaction 202, a person 101 states that he has a craving. The system 100 classifies this inquiry or received statement from the person using the natural language machine learning models 150a-150f illustrated in FIG. 1.

In this example, the system 100 categorizes the inquiry as a warning sign. The system 100 then provides scripted information regarding dialogue for warning sign behavior from the person 101. In some implementations, the scripted information has been adapted for the particular person 101 based on profile information and other information obtained from previous interactions with the system 100. The system 100 uses additional machine learning models to determine the appropriate response to the person's initial statement and predict whether a person will relapse.

In this example, after categorizing the person's statement as a warning sign, the system 100 determines that the person 101 needs accountability in order to maintain sobriety. Therefore, the system 100 asks the person 101 whether the person has attended a sobriety meeting today.

In a second interaction 204, the person 101 answers the system's 100 query regarding whether he has attended his sobriety meeting. The person 101 then follows up with a question regarding the time of the meeting. The system 100 then answers the question and offers to set a reminder in order for the person 101 to leave on time.

In a final interaction 206, the system 100 responds to the person's positive response to set a timer and sends directions to the meeting to the person's car.

The system 100 can know that the person 101 is in the car on the way to the meeting, has arrived at the meeting, or is leaving the meeting using any of the person's 101 networked-devices, e.g., devices that are connected using the Internet, Bluetooth, NFC, cellular network, or other network. These devices can include wearables, smartphones, and wireless-enabled sensors in the car. Once the system 100 determines that the person 101 followed the recovery plan and attended the meeting, the system can reward the person, e.g., using digital currency such as Noiacoin. The Noiacoin (NCN), like any accepted cryptocurrency can be traded on any supported exchange for any supported crypto trading pair, e.g., NCN/BTC, NCN/ETH, and eventually for fiat. Award/and or credits may be given towards time served, or requirements handed out by courts, therapists, rehabilitation centers, etc.

Additional rewards include badges and progression up a leaderboard with other users of the system 100, e.g., friends of the person struggling with addiction.

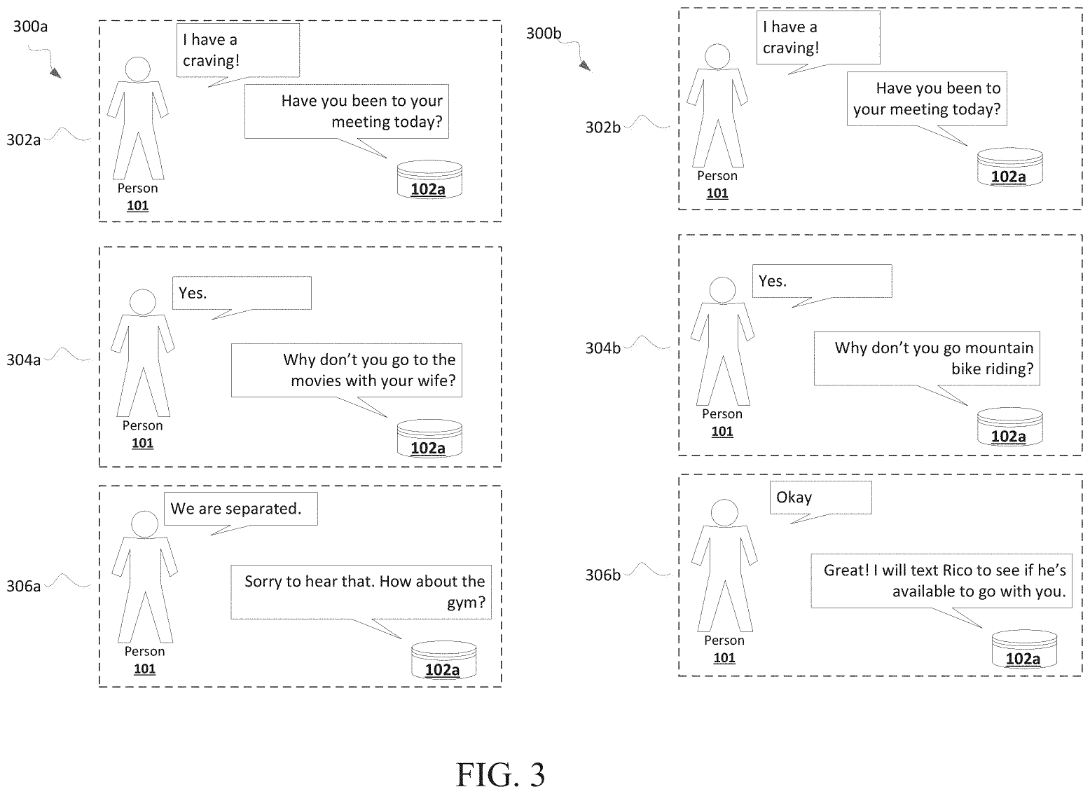

FIG. 3 illustrates examples of two dialogues between a person 101 and the system 100 that have been tailored to the person based on user profile information and previous interactions. Dialogue 300a represents a conversation that occurs with the person at a date prior to the conversation occurring in Dialogue 300b. In Dialogue 300a, after the person 101 has stated that he has in fact attended his meeting for the week, and yet is still having cravings, the system 100 suggests that he do something with his wife 304a. However, in the next interaction 306a, the system 100 learns from the person that he and his wife are separated. The system 100 then makes an alternative suggestion.

Dialogue 300b illustrates that the system 100 has learned from the previous interaction in Dialogue 300a so that when the person 101 states that he has a craving and has already attended his meeting, the system 100 no longer suggests activities with the person's wife. Instead, the system 100 can pull information from the person's profile that is saved in a database in the system 100, containing answers to the initial registration questions as well as pertinent information that the system 100 has learned about the person 101 over time. In this example, the system 100 tailors the response to the person 101 by knowing that the person 101 likes to mountain bike ride and suggests this activity. The system 100 can also have access to the person's contact information from other networked devices and then suggest a friend to join the person 101 on the ride.

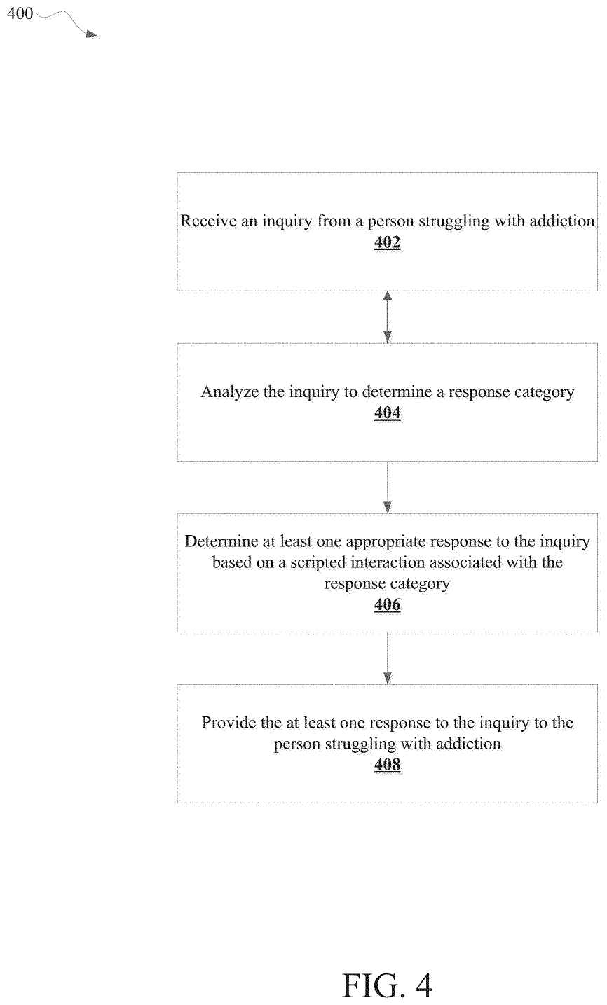

FIG. 4 is a flowchart of an example process 400 for providing a digital virtual sponsor to facilitate addiction recovery. For convenience, the process 400 will be described as being performed by a system of one or more computers, located in one or more locations, and programmed appropriately in accordance with this specification. For example, a digital virtual sponsor system, e.g., the digital virtual sponsor system 100 of FIG. 1, appropriately programmed, can perform the process 400.

First, the system 100 receives an inquiry from a person struggling with addiction 402. The system analyzes the inquiry to determine a response category 404. As described above, the response category can be any category determined by natural language machine learning models e.g., information, motivation, or warning-sign. The system then determines at least one appropriate response to the inquiry based on a scripted interaction associated with the response category 406. The system 100 uses machine learning, user profiles, the knowledge base, and previous interactions with the user to determine the appropriate response to the given inquiry. The machine learning and knowledge base elements of the system 100 aggregate the actual experiences of a large number of recovering persons and their responses to determine specific recommendations and responses. The user profile and previous interactions of the user with the system provide personalized information about the user to the machine learning subsystem 105, which uses the personalized information to identify responses that are most effective for individuals having the same attributes as that particular user.

The system then provides the at least one response to the inquiry to the person struggling with addiction. The response can be provided using the communication platform that the person used to communicate with the system or any number of other networked devices that are programmed to accept a response.

The response may include suggestions or activities for avoiding addictive behavior. The system can incentivize positive behavior that encourages recovery by providing rewards for following the suggestions, participating in the activities, or otherwise accomplishing recovery goals. For example, if the system 100 determines that the person struggling with addiction has taken the suggestions or done the recommended activities, the system can reward the person with digital currency or other incentives. The digital currency may be provided in different amounts depending on the amount the system 100 allocates for each suggestion, activity, or personal accomplishment.

In some implementations, the system 100 can initiate communication with a person struggling with addiction to ensure that the person is maintaining sobriety.

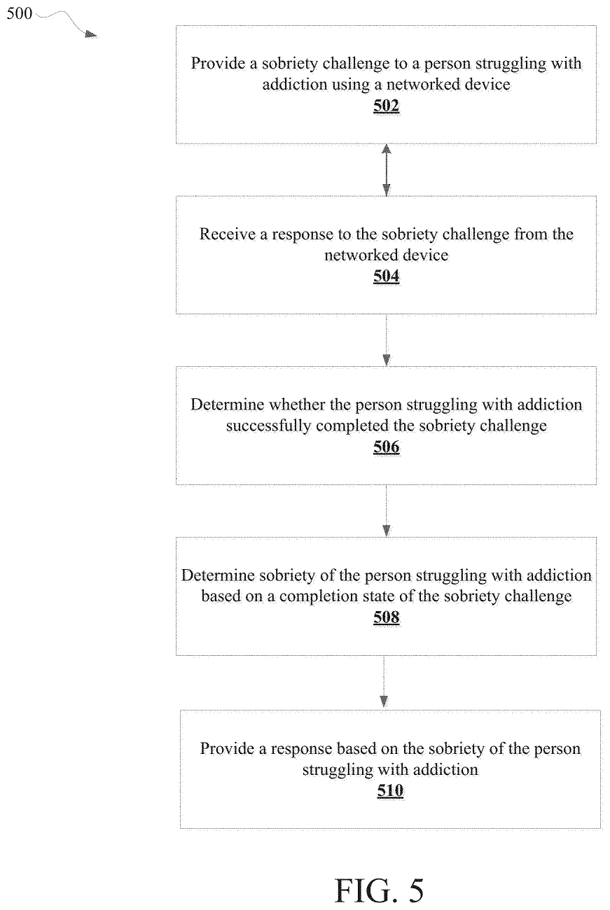

FIG. 5 is a flowchart of an example process 500 for determining sobriety of a person struggling with addiction. For convenience, the process 500 will be described as being performed by a system of one or more computers, located in one or more locations, and programmed appropriately in accordance with this specification. For example, a digital virtual sponsor system, e.g., the digital virtual sponsor system 100 of FIG. 1, appropriately programmed, can perform the process 500.

The system 100 provides a sobriety challenge to the person struggling with addiction using a networked device 502. The sobriety challenge can be a question that the person needs to answer, e.g., “Have you attended your meeting?” or “Have you talked with your sponsor?” The sobriety challenge can also be a puzzle, e.g., a brain teaser, Sudoku, or other mentally challenging activity that can be issued digitally and returned to the system. As such, the sobriety challenge can be a dexterity or thought task that measures a person's attentiveness and responsiveness. Therefore, a wide variety of challenges may be used that allows the system to determine if a user is clean and sober or if the user has started using again. The sobriety challenge can be issued on regular intervals such as daily, hourly, twice a day, or some other predefined time period, or in response to an interaction (a conversation or engagement with an activity) that the user had with the system in order to ensure sobriety of an individual.

The system 100 then receives a response to the sobriety challenge 504. In some implementations, the sobriety challenge will have a time limit for response. Either the person to whom the sobriety challenge was sent will return the challenge to the system in the time limit or the system will register a timeout response. The system 100 determines whether the person successfully completed the sobriety challenge 506. The system 100 can do this by comparing the sobriety challenge answer with a correct answer if the challenge was a puzzle, checking with the meeting logs or sponsor to determine if the person went to a meeting or met with the sponsor, or using other records to verify the response of the person. The system can obtain information to verify the sobriety challenge answer using networked devices, e.g., IoE devices belonging to the person or the person's contacts.

After determining whether the person struggling with addiction successfully completed the sobriety challenge, the system determines the sobriety of the person based on the completion state of the sobriety challenge 508. The system can then provide a response based on the sobriety of the person struggling with addiction.

If the person successfully completed the sobriety challenge, the system determines that the person is sober. The system may then reward the person with digital currency or some other incentive. The system may also schedule a second sobriety challenge for a time period in the future.

If the person does not successfully complete the challenge or the challenge times out before the person responds, the system determines that the person may not be sober. People struggling with addiction often cheat for a few days before completely relapsing. The system can use a threshold amount of time of failed challenges or unresponsiveness to determine that a user is in danger of relapsing or has relapsed. The system may then try to engage the person in dialogue to determine how to help the person to follow recovery steps. In some implementations, the system determines the events that occurred in the person's life prior to relapsing in order to predict relapses in the future, and to help avoid such relapses again through new suggestions, therapy, information, or other recovery techniques.

The system may also try to contact people from the person's contact list in order to have the contacts encourage the person to follow the recovery steps. The system may also take measures to stop the person from spiraling into addiction, e.g., canceling credit cards, disabling vehicles, locking cabinets, and freezing bank accounts, notifying appropriate persons, e.g., family, friends, officials, etc. In some implementations, the system requires opt-in permissions from the user or mandated by a third-party and agreed to by the user prior to taking such measures, collecting user data, and/or notifying others of the user's progress.

The system can provide resources and information regarding how to help the person in his or her current state if they have slipped back into addiction. For example, if the person is homeless or jobless, the system can suggest temporary housing or jobs in the person's skillset. The system can provide any number of resources and accountability to support the person's recovery.

In addition to system-issued challenges, the system 100 can allow friends or contacts of the person struggling with addiction to pose recovery challenges and activities in which the participants compete against each other. A leaderboard can indicate the progress of all participants as compared to one another and show winners of challenges.

With consent of registered users, the system can additionally collect data for research and analytical analysis. This data can be used to determine a root cause for relapse among individual recovering persons.

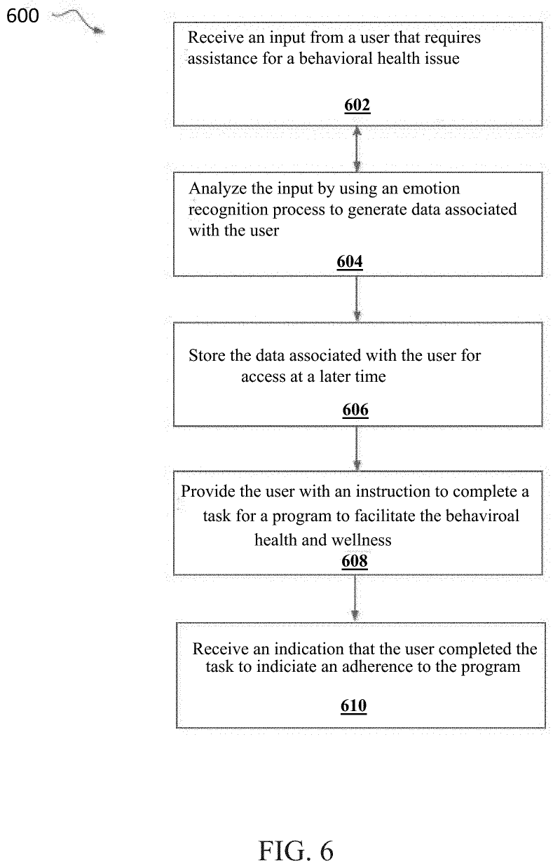

In an alternative embodiment, FIG. 6 illustrates a flowchart of an example process 600 of using digital virtual sponsor system 100 to facilitate behavioral health and wellness of person 101. It shall be appreciated that behavioral health and wellness pertains to addressing any behavioral health issues including, but not limited to, addiction, post-traumatic stress disorder (PTSD), depression and other mental health disorders. Process 600 is designed to more effectively provide for behavioral health and wellness of person 101 based on the recognition that individuals in recovery experience a wide range of emotions, many times within the same day.

An assessment of these emotions and understanding of the source of these emotions provide caretakers, counselors and therapists additional insight into the behavioral health of addicts or individuals diagnosed with mental health disorders. In one embodiment, machine learning subsystem 105 of digital virtual sponsor system 100 may implement WingMan for Addiction, which is an application that is executed by a processor of machine learning subsystem 105. WingMan for Addiction is configured to detect one of a plurality of emotional states of a user (e.g., person 101) who interacts with the application. As a result of performing the described emotion recognition process, digital virtual sponsor system 100 is able to more effectively provide behavioral health and wellness responses that are tailored to the needs of person 101, thereby facilitating improved success rates of the recovery and/or wellness process.

In one embodiment, process 600 is performed with the following exemplary steps. In step 602, system 100 receives an input from person 101 who requires assistance with a behavioral health issue. System 100 receives the input via communication platform 103 by use of any one or combination of communication device 102a, smartphone 102b or computing device 102c. The received input from person 101 generally comprises a string of one or more words, letters, digits and punctuation in a text chat or voice chat, which is transmitted by communication platform 103 over network 130 to machine learning subsystem 105. In one embodiment, person 101 speaks or types questions and/or comments into communication platform 103, and the WingMan application continues the interaction by providing a corresponding response to person 101. Alternatively, other interactions between system 100 and person 101 may include listening to inspirational audio recordings, meditations and/or videos, or reading recovery stories and writing in a journal.

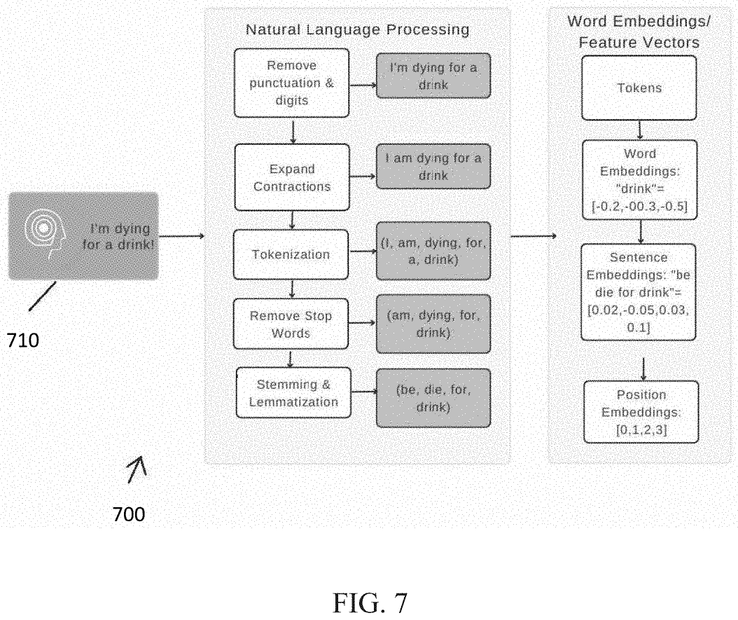

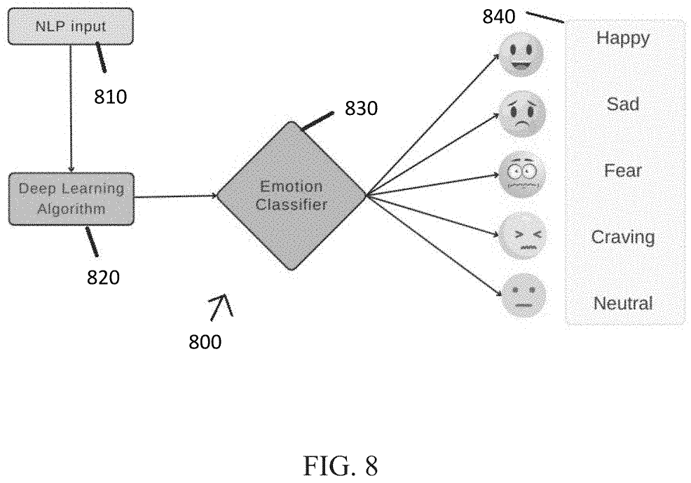

In step 604, system 100 analyzes the received input by using an emotion recognition process to generate data associated with person 101. In one embodiment according to FIGS. 7-8, the emotion recognition process of step 604 includes natural language processing 700 and emotion determination processing 800, which are performed to generate the data associated with person 101.

As depicted in FIG. 7, system 100 performs natural language processing 700 on the received input corresponding to string 710. String 710 comprises any number and combination of words, punctuation and digits, e.g., “I'm dying for a drink!”. In natural language processing 700, system 100 performs a plurality of preprocessing steps that include the following exemplary steps: (1) Remove any punctuation and digits from string 710, e.g., remove commas, periods, quotation marks, apostrophes, numbers, and the like; (2) Expand any contractions present in string 710, e.g., modify words such as “don't” to be “do not” to capture the full meaning of the text; (3) Perform a tokenization on the words in string 710 to divide the input into groupings of letters, which allow for an easier conversion to numerical values; (4) Remove any present stop words in string 710, e.g., remove commonly used words that do not have significant meaning such as “by”, “or”, “and”, etc.; (5) Perform stemming and lemmatization on the words in string 710, e.g., reduce words to their roots, such as changing “starting” to “start” and modifying verbs to their base or dictionary form (e.g., “is” to “be”); (6) Perform word embeddings on string 710, e.g., change words with similar meaning to have a similar representation; (7) Perform sentence embeddings on string 710, e.g., represent entire sentences and their semantic information as vectors; and (8) Perform position embeddings on string 710, e.g., capture the input order of the words to retain a greater meaning.

In alternative embodiments, it shall be appreciated that the preprocessing steps of natural language processing 700 can be modified to remove steps, add steps, and/or change the order of steps that are performed.

As depicted in FIG. 8, after the preprocessing steps of natural language processing 700 are performed on string 710, system 100 performs emotion determination processing 800 on the resultant natural language processing input 810 generated from string 710. Specifically, system 100 converts the remaining words in natural language processing input 810 into numerical values as an input for the neural networks of machine learning subsystem 105. System 100 feeds the numerical input to Deep Learning Algorithm 820 of machine learning subsystem 105, which uses a classification algorithm corresponding to emotion classifier 830 to recognize one or more emotions 840 experienced by person 101. Emotions 840 comprise a plurality of categories of emotions that include, but are not limited to, feelings of happiness, sadness, fear, craving and being neutral. However, it shall be appreciated that emotions 840 can include any alternative number and type of emotions.

In one embodiment, system 100 analyzes the numerical values associated with string 710 using a neural network of machine learning subsystem 105 to classify the received input from person 101 into one of a plurality of emotions 840, on a basis of stored historical data pertaining to strings of prior user conversations that are each classified into one of the emotions 840. The historical data is stored in a neural network in any storage device of system 100 and corresponds to training data from a large sample of at least thousands of prior conversations of users of system 100, which indicate common words and phrases, and their corresponding associated classifications into at least one of the plurality of emotions 840.

For example, the saved historical data comprises a list of common words including, but not limited to, “life”, “happy”, “great”, “sober”, “help”, “right”, “agree”, “grateful”, “recovery”, “clean”, “nice” and “positive”, which are associated with and classified into the emotion of happiness. Further, the saved historical data comprises a list of common words including, but not limited to, “depressed”, “like”, “sad”, “talk”, “hurt”, “die”, “bad”, “sick”, “drink”, “doctor”, “rough” and “guilt”, which are associated with and classified into the emotion of sadness. Further, the saved historical data comprises a list of common words including, but not limited to, “stress”, “help”, “anxious”, “friend”, “addiction”, “talk”, “tired”, “motivation”, “frustrated”, “inspiration”, “angry” and “alcoholic”, which are associated with and classified into the emotion of fear. Further, the saved historical data comprises a list of common words including, but not limited to, “drink”, “use”, “craving”, “drug”, “relapse”, “struggle”, “tonight”, “smoke”, “pill”, “dealer”, “bar” and “gamble”, which are associated with and classified into the emotion of a craving.

During emotion determination processing 800, system 100 is able to effectively analyze the numerical values of the words and entire sentence in string 710 from person 101 using a neural network created from stored historical data including common words and corresponding classified emotions, to determine a match that indicates person 101 presently experiences one or more of the plurality of emotions 840. As a result, emotion determination processing 800 is advantageous because it allows system 100 to determine one or more emotions of person 101, based on actual data of conversations and corresponding detected emotions of prior addicts and other persons with behavioral health issues. The advantage of determining these emotions is that the system 100 and behavioral health counselors providing assistance to person 101 can more directly and effectively address the specific issues being experienced by person 101.

In one embodiment, system 100 is configured to employ voice analytics to evaluate audio frequency and volume levels in a voice input of person 101 corresponding to string 710. This processing of a voice input can confirm specific emotions of person 101, even if a textual analysis of the same string to determine corresponding emotions is inconclusive.

In one embodiment, emotion classifier 830 is built based on a multi-channel convolutional neural network (CNN) and Long Short-Term Memory (LSTM). These tools use the input of numerical values created by natural language processing 700. The CNN uses a window (referred to as convolution) that slides over the numerical values associated with the words in the incoming sentence with an emphasis on a subset of the input data, providing a more narrow focus. This creates a data set of the numerical values of words that is most relevant to detecting emotion. The LSTM identifies relationships between the numerical values associated with words that occurred earlier in the input with words that occurred later in the input. The combination of numerical values for the most important words and numerical values of the sequence of words in the user's input, enables the classification of the converted incoming sentence/string (the result of applying Natural Language Processing) into one or more of the predetermined emotions or classes.

At step 606 of process 600 according to FIG. 6, system 100 saves the generated data associated with person 101 in any storage device, which can be accessed at a later time by anyone with access to the system, such as person 101, caretakers, counselors, therapists, and the like. The saved data associated with person 101 can include any data associated with string 710, which is generated during natural language processing 700 and emotion determination processing 800.

At step 608, system 100 provides an instruction via communication platform 103 for person 101 to complete a task as part of a program to facilitate behavioral health and wellness. The task can include, but is not limited to, a sobriety challenge as previously described, or a request for attendance at a meeting, workshop, seminar, group therapy session or training class. In one embodiment, system 100 recommends a specific task for person 101 to complete in the program based on the stored data that is generated from natural language processing 700 and emotion determination processing 800. Alternatively, system 100 can recommend a task of the program to person 101 based on other collected usage data of system 100 including, but not limited to, information related to sentiment and emotion, location tracking, completion of activities that were prescribed by an accountability person or organization, assessment surveys, sobriety challenges, and measures of overall improvement.

At step 610, system 100 receives an indication that person 101 has completed the task to indicate an adherence to the behavioral health and wellness program. System 100 can be notified of the completion of the task either directly from person 101 via communication platform 103, or indirectly from another party such as a caretaker, counselor or therapist with access to system 100. In one embodiment, system 100 subsequently issues a non-fungible token (NFT) in blockchain ecosystem 106 by using the Proof of Attendance Protocol (POAP) to indicate a permanent record of the task that is completed by person 101. Using a digital wallet, person 101 may display the NFT as verified proof of attendance to accountability persons and/or organizations who wish to see evidence of the adherence by person 101 to a behavioral health and wellness program.

In one embodiment, system 100 comprises a portal on communication platform 103 to allow caregivers and other interested parties to view/access any stored data associated with the activities and emotional trends of users on a group level. For example, the portal can display a specified group's activity levels and emotions over the past week, month, or quarter. A person can select an individual user of the WingMan application via the portal, and request additional details of the selected user. This information accessed through the portal allows caregivers and health professionals to measure the effectiveness of current prescribed treatment and counseling, in order to improve overall outcomes with patients.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array), an ASIC (application-specific integrated circuit), a GPU (graphical programming unit), or a CPU (central processing unit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be exercised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. In some circumstances, quantum computing/processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method of using a digital virtual sponsor to facilitate behavioral health and wellness of a user, the method comprising:
receiving an input from the user requiring assistance for a behavioral health issue;
analyzing the input from the user by using an emotion recognition process to generate data associated with the user;
storing the data associated with the user for access at a later time;
providing the user with an instruction to complete a task as part of a program to facilitate the behavioral health and wellness of the user, on a basis of the data associated with the user; and
receiving an indication that the user completed the task to indicate an adherence to the program by the user.

2. The method of claim 1, further comprising issuing a non-fungible token in a blockchain ecosystem by using a Proof of Attendance Protocol (POAP) to indicate a permanent record of the task that is completed by the user.

3. The method of claim 1, wherein the input from the user corresponds to a string associated with a conversation of the user, the string being formed by a plurality of words, a punctuation and one or more digits.

4. The method of claim 3, wherein analyzing the input by using the emotion recognition process to generate the data associated with the user comprises:
performing natural language processing on the string;
after natural language processing is performed on the string, converting the plurality of words of the string into numerical values;
analyzing the numerical values associated with the string using a neural network to classify the input of the user into one of a plurality of emotions, on a basis of stored historical data pertaining to strings of prior user conversations that are each classified into one of the emotions in the plurality of emotions; and
determining that the user presently experiences one of the plurality of emotions based on the classified input of the user into the one of the plurality of emotions.

5. The method of claim 4, wherein the plurality of emotions comprises feelings of happiness, sadness, fear, craving and being neutral.

6. The method of claim 4, wherein performing natural language processing on the string comprises:
removing the punctuation and the one or more digits from the string;
expanding a contraction in the string;
performing a tokenization on the plurality of words in the string;
removing a stop word in the string;
performing stemming and lemmatization on the plurality of words in the string; and
performing word embeddings, sentence embeddings and position embeddings on the string.

7. The method of claim 1, wherein the task of the program is a sobriety challenge, or attendance at a meeting, workshop, seminar, group therapy session or training class.

8. The method of claim 1, further comprising continuously collecting usage data of the digital virtual sponsor corresponding to a progress of the user in the program over a period of time, and displaying the usage data on a display interface.

9. A system for providing a digital virtual sponsor to facilitate behavioral health and wellness of a user, the system comprising:
a processor to execute a program; and
a memory to store the program which, when executed by the processor, the processor performs processes of,
receiving an input from the user requiring assistance for a behavioral health issue;
analyzing the input from the user by using an emotion recognition process to generate data associated with the user;
storing the data associated with the user for access at a later time;
providing the user with an instruction to complete a task as part of a program to facilitate the behavioral health and wellness of the user, on a basis of the data associated with the user; and
receiving an indication that the user completed the task to indicate an adherence to the program by the user.

10. The system of claim 9, wherein the processor further performs a process of:
issuing a non-fungible token in a blockchain ecosystem by using a Proof of Attendance Protocol (POAP) to indicate a permanent record of the task that is completed by the user.

11. The system of claim 9, wherein the input from the user corresponds to a string associated with a conversation of the user, the string being formed by a plurality of words, a punctuation and one or more digits.

12. The system of claim 11, wherein in analyzing the input by using the emotion recognition process to generate the data associated with the user, the processor further performs processes of:
performing natural language processing on the string;
after natural language processing is performed on the string, converting the plurality of words of the string into numerical values;
analyzing the numerical values associated with the string using a neural network to classify the input of the user into one of a plurality of emotions, on a basis of stored historical data pertaining to strings of prior user conversations that are each classified into one of the emotions in the plurality of emotions; and
determining that the user presently experiences one of the plurality of emotions based on the classified input of the user into the one of the plurality of emotions.

13. The system of claim 12, wherein the plurality of emotions comprises feelings of happiness, sadness, fear, craving and being neutral.

14. The system of claim 12, wherein the processor performs the natural language processing on the string by performing processes of:
removing the punctuation and the one or more digits from the string;
expanding a contraction in the string;
performing a tokenization on the plurality of words in the string;
removing a stop word in the string;
performing stemming and lemmatization on the plurality of words in the string; and
performing word embeddings, sentence embeddings and position embeddings on the string.

15. The system of claim 9, wherein the task of the program is a sobriety challenge, or attendance at a meeting, workshop, seminar, group therapy session or training class.

16. The system of claim 9, wherein the processor further performs processes of:
continuously collecting usage data of the digital virtual sponsor corresponding to a progress of the user in the program over a period of time; and
displaying the usage data on a display interface.
